(19) United States
(12) Patent Application Publication
(10) Pub. No.: US 2021/0000898 A1
(43) Pub. Date: Jan. 7, 2021

(54) COMPOSITION HAVING ERYTHROPOIETIN-INDUCING ACTIVITY, AND METHOD FOR PRODUCING SAME

(71) Applicant: Fumio Kobayashi, Ibaraki (JP)

(72) Inventors: Fumio Kobayashi, Ibaraki (JP); Masayoshi Saito, Chiba (JP)

(73) Assignee: Fumio Kobayashi, Ibaraki (JP)

(21) Appl. No.: 17/040,877

(22) PCT Filed: Mar. 25, 2019
(86) PCT No.: PCT/JP2019/012425

§ 371 Date: Sep. 23, 2020

(30) Foreign Application Priority Data
Mar. 26, 2018 (JP) 2018-058380

(51) Int. Cl. A61K 36/068; A61P 7/00; A23L 33/10; A23L 31/00; A23L 2/52

(52) U.S. Cl. CPC A61K 36/068; A61P 7/00; A23L 33/10; A23V 2002/00; A23L 2/52; A61K 2236/11; A61K 2236/333; A23L 31/00

(57) ABSTRACT

A composition that has an erythropoietin-inducing activity and can be used as a drug or a food or a beverage is produced by using a culture of Cordyceps militalis or an extract from the culture. In one example of the method for producing such a composition, solid medium containing a protein and a grain is used. In another example of the method, liquid medium containing glucose, yeast extract, rice bran (KOMENUKA), sake lees, soybean powder, sodium aspartate, an inorganic substance and water is used. This composition can be used for the prevention or treatment of anemia.

### TECHNICAL FIELD

The present invention relates to a composition comprising culture of Cordyceps militalis that has an erythropoietin-inducing activity or an extract from the culture, and a method for producing the composition.

### BACKGROUND ART

After the second world war, in Japan income level has increased and dietary life has been improved through the high economic growth period. At the same time, the crisis rate of diabetes has also been increasing because of ingestion of caloric rich foods, and the incidence rates of complications derived from diabetes have also been increasing year by year. The incidence of kidney diseases among such complications is particularly remarkable. The number of patients of renal anemia and that of renal dialysis tend to increase year by year.

The decrease of renal function steers to lack of erythropoietin which is one of hematopoietic factors and which is mainly produced in kidney, and as the result, renal anemia occurs. In recent years, various sports have become common, and as the result, not only professional players but also amateur players are active in various fields. Various sports give pleasure. On the other hand, if one persists in record or victory or defeat, exercise of one's body and training also become hard year by year. The actual circumstances are said that there are many anemia patients even if the patients are athletes.

By the way, in the traditional Chinese medical science, mushrooms of genera Cordyceps (fungi of Cordyceps), fungus of Cordyceps sinensis (Berkeley) Saccardo) among them, have been used heretofore. However, wild type of it has reached near ecological extinction. Therefore, other mushrooms of genera Cordyceps have been studied about their culturing method, bioactivity, effective components that indicate the bioactivity, and the like to review their availability as substitutes of Cordyceps sinensis.

Patent literature 1 discloses an artificial culturing method of a fruit body of Cordyceps militaris (Vuill.) and discloses that the fruit body contains adenosine and cordycepin having an antitumor activity as its bioactive substances. Patent literature 1 discloses a culture solution of mycelia of Cordyceps militaris (Vuill.) (spawn for culture) containing glucose, peptone, a potato extract, KH₂PO₄, MgSO₄.7H₂O and a distilled water, and culture medium of a fruit body of Cordyceps militaris (Vuill.) containing OKARA (residual meal which is obtained by squeezing soymilk in the production of soybean curd (tohu)), rice bran (KOMENUKA) and soybean meal.

Patent literature 2 describes a method for producing a dry powder of culture derived from Isaria japonica Yasuda or Isaria sinclairii (Berk.) Lloyd which belongs to genus Cordyceps and describes that culture or an extract, or a processed material of these mushrooms contains an enhancer of cytokine production. Patent literature 2 also describes that the culture of Isaria japonica Yasuda or Isaria sinclairii (Berk.) Lloyd has an enhancing effect of production of GM-CSF among cytokines, and that the GM-CSF has also the nature to increase a hematopoietic function of leukocyte. Also, patent literature 2 discloses liquid medium containing glucose and dry yeast as liquid medium of Isaria japonica Yasuda or Isaria sinclairii (Berk.) Lloyd for large-scale culture.

Patent literature 3 discloses a composition characterized by containing a culture filtrate of cultured mycelia of Cordyceps militaris and/or a solvent extract from the mycelia. Patent literature 3 describes that this composition has a combination of a mild cardiac action and a bronchodilator or antitussive action. Also, patent literature 3 discloses M20Y2 medium (the composition in 100 mL: malt extract 2 g, yeast extract 0.2 g; pH5.5) as liquid culturing medium of Cordyceps militaris.

### PRIOR ART LITERATURES

### Patent Literatures

Patent literature 1: Bulletin of Japanese patent No. 5800211
Patent literature 2: Bulletin of Japanese patent laid-open No. 2005-104848
Patent literature 3: Bulletin of Japanese patent laid-open No. Hei 8-12588

### SUMMARY OF INVENTION

### Problem to be Solved by Invention

Genera Cordyceps have a potential for containing various bioactive substances. Therefore, the purpose of the present invention is to find new usefulness of Cordyceps militaris by studying a culturing method of Cordyceps militaris among genera Cordyceps, and by studying an extraction method of bioactive substances from its culture and bioactivities of the substances that are contained in the culture or the extract.

### Means for Solving Problem

Regarding Cordyceps militaris, the present inventors have studied its culturing method, and also an extraction method of bioactive substances from the culture and bioactivities of the substances that are contained in the culture or the extract. As a result, they have accomplished the present invention.

Namely, the present invention relates to the following compositions:

(1) A composition that has an erythropoietin-inducing activity comprising culture of Cordyceps militalis or an extract from the culture;
(2) The composition described in (1), wherein the culture of Cordyceps militalis is culture of mycelia;
(3) The composition described in (1) or (2), wherein the culture of Cordyceps militalis comprises culture that has been cultured by using solid medium;
(4) The composition described in (3), wherein the solid medium comprises a mite's body;
(5) The composition described in any one of (1) to (4), which further has an activity of increase of leucocyte's number; and
(6) The composition described in any one of (1) to (5), which is a drug, or a food or a beverage.

The present invention also relates to the following methods for producing a composition that has an erythropoietin-inducing activity:

(7) A method for producing a composition that has an erythropoietin-inducing activity, comprising step (I) comprising inoculating spawn of Cordyceps militalis to solid medium comprising a protein and a grain and culturing mycelia;
(8) The producing method described in (7), which further comprises step (II) comprising obtaining an extract comprising a component that has an erythropoietin-inducing activity from solid culture of Cordyceps militalis that has been obtained through the step (I);
(9) The producing method described in (7) or (8), wherein the step (II) comprises a step comprising carrying out warming extraction and heating extraction by using ethanol having a concentration of 20 to 50% by weight; and
(10) The producing method described in any one of (7) to (9), wherein the protein comprises a protein derived from a mite's body.

The present invention further relates to the following methods for producing a composition that has an erythropoietin-inducing activity:

(11) A method for producing a composition that has an erythropoietin-inducing activity, comprising step (1) comprising inoculating spawn of Cordyceps militalis to liquid medium comprising glucose, yeast extract, rice bran (KOMENUKA), sake lees, soybean powder, sodium aspartate, an inorganic substance and water and culturing mycelia;
(12) The producing method described in (11), which further comprises step (2) comprising obtaining an extract comprising a component that has an erythropoietin-inducing activity from liquid culture of Cordyceps militalis that has been obtained through the step (1); and
(13) The producing method described in (12), wherein the step (2) comprises a step comprising adding ethanol to liquid culture of Cordyceps militalis that has been obtained through the step (1) and carrying out warming extraction and heating extraction, wherein the ethanol concentration of liquid comprising the liquid culture and the ethanol is 20 to 50% by weight.

### Effects of Invention

The composition of the present invention has an erythropoietin-inducing activity and is high in safety. Therefore, it is valid for prevention or treatment of renal anemia as a drug, or a food or a beverage.

Regarding the composition of the present invention which has also an activity of increase of leucocyte's number, in combination with the erythropoietin-inducing activity, more remarkable hematopoietic effect can be expected.

The composition of the present invention can be prepared from culture of mycelia of Cordyceps militalis, and the mycelia of Cordyceps militalis can be artificially cultured. Thus, raw materials can be stably supplied.

There also exists an effect that the extraction of active components from culture of Cordyceps militalis can be performed under relatively mild conditions.

### BRIEF DESCRIPTIONS OF FIGURES

FIG. 1 FIGS. 1-1 to 1-4 are reports of analysis results of ITS-5, 8S rDNA of Cordyceps militalis FT26K3.

FIG. 2 FIGS. 2-1 to 2-7 are reports of analysis results of ITS-5, 8S rDNA of Cordyceps militalis KT16514.

FIG. 3 is a graph showing erythropoietin concentrations of blood plasmas, to which heparin was added and which were obtained from mice to which CM-A had been administered.

FIG. 4 is a graph showing erythropoietin concentrations of blood plasmas, to which heparin was added and which were obtained from mice to which CM-B had been administered

FIG. 5 is a graph showing erythropoietin concentrations of blood plasmas, to which heparin was added and which were obtained from mice to which CM-A had been administered.

FIG. 6 are graphs showing erythropoietin concentrations, numbers of erythrocyte, hemoglobin concentrations and hematocrit levels in bloods before and after administrations of CM-A to human (period of administration: 1 week).

FIG. 7 are graphs showing variations of erythropoietin concentration, number of erythrocyte, hemoglobin concentration and hematocrit level in blood by administration of CM-A to human (period of administration: 4 months).

### ILLUSTRATIVE EMBODIMENTS TO PERFORM INVENTION

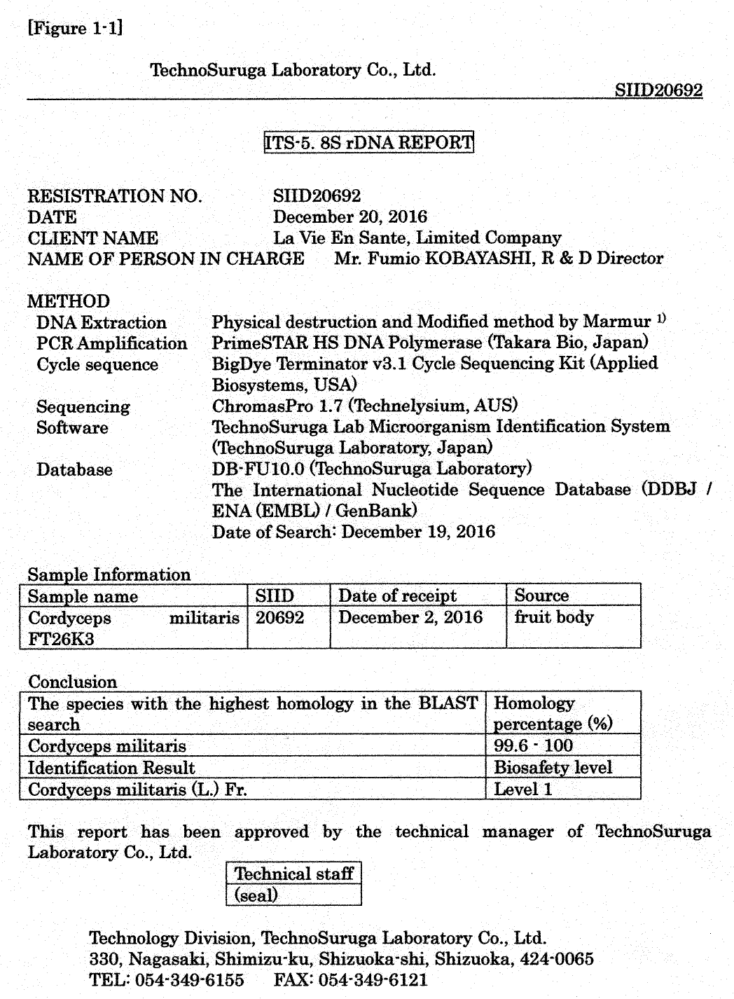

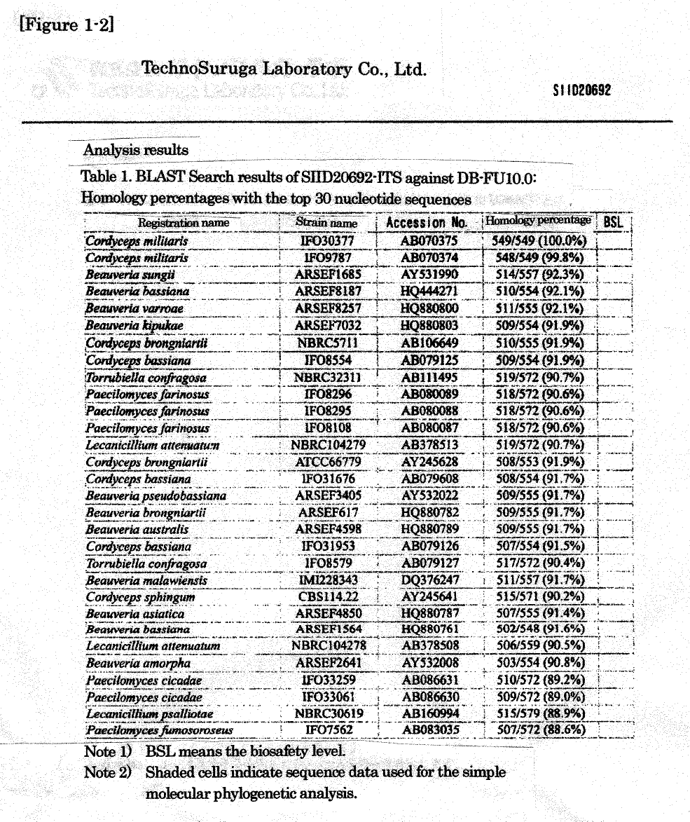

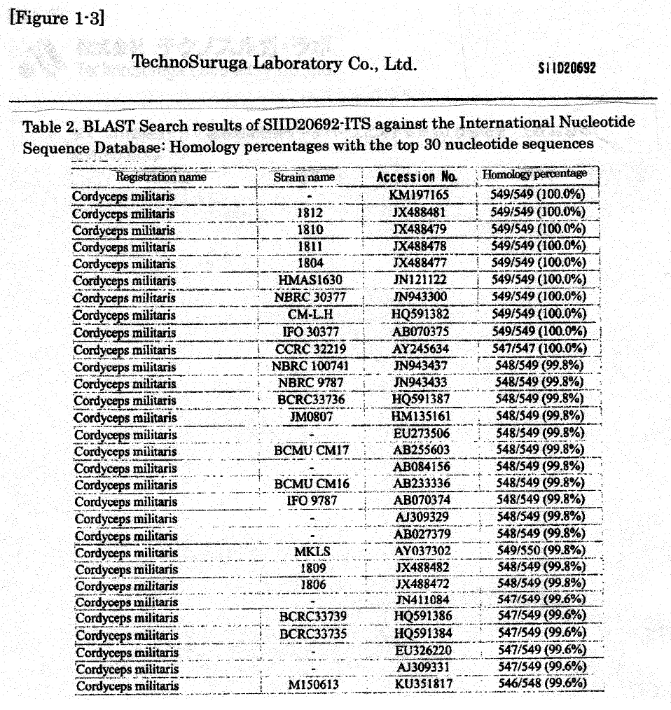

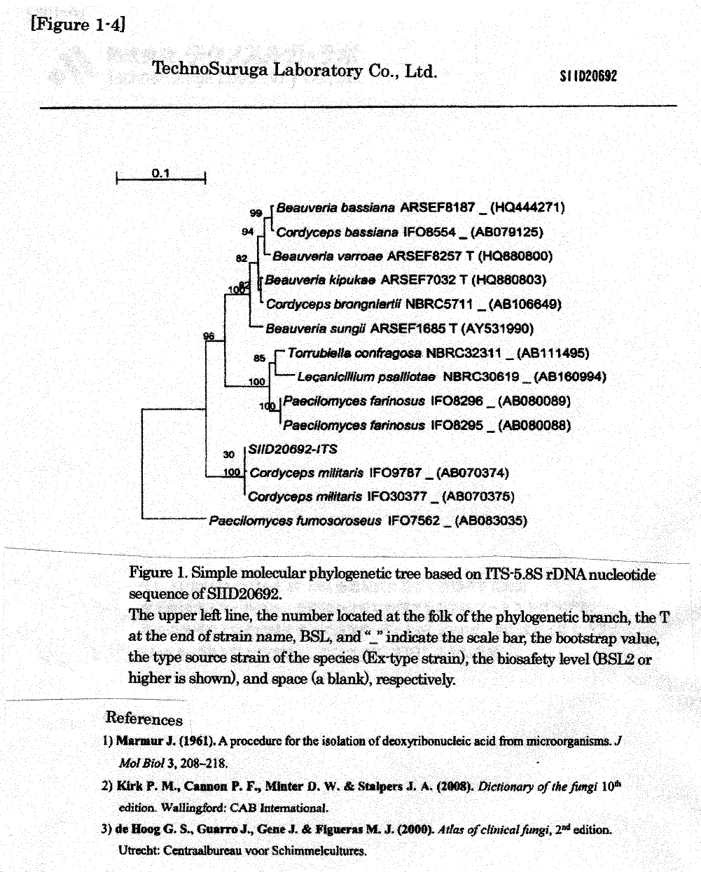

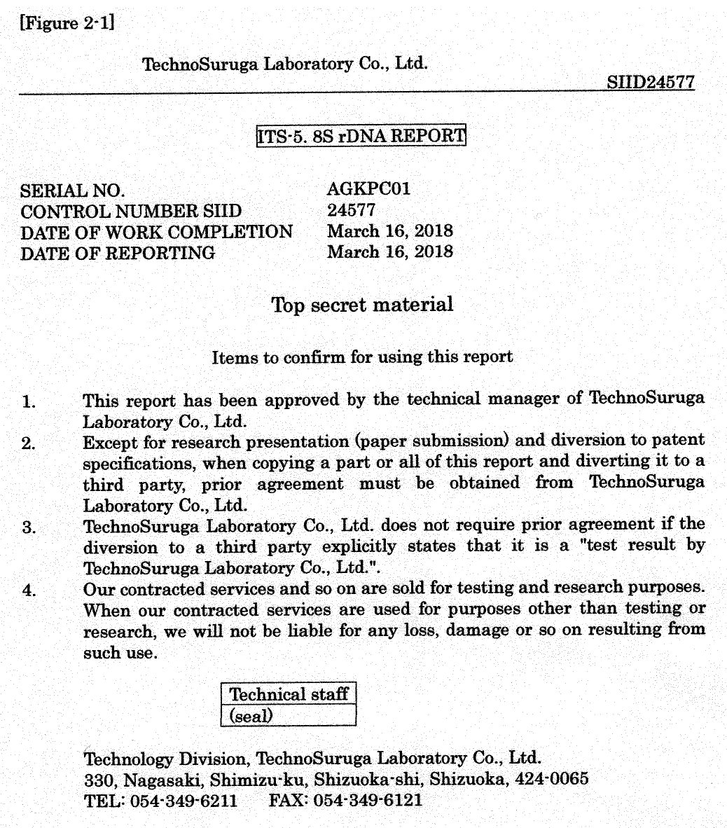

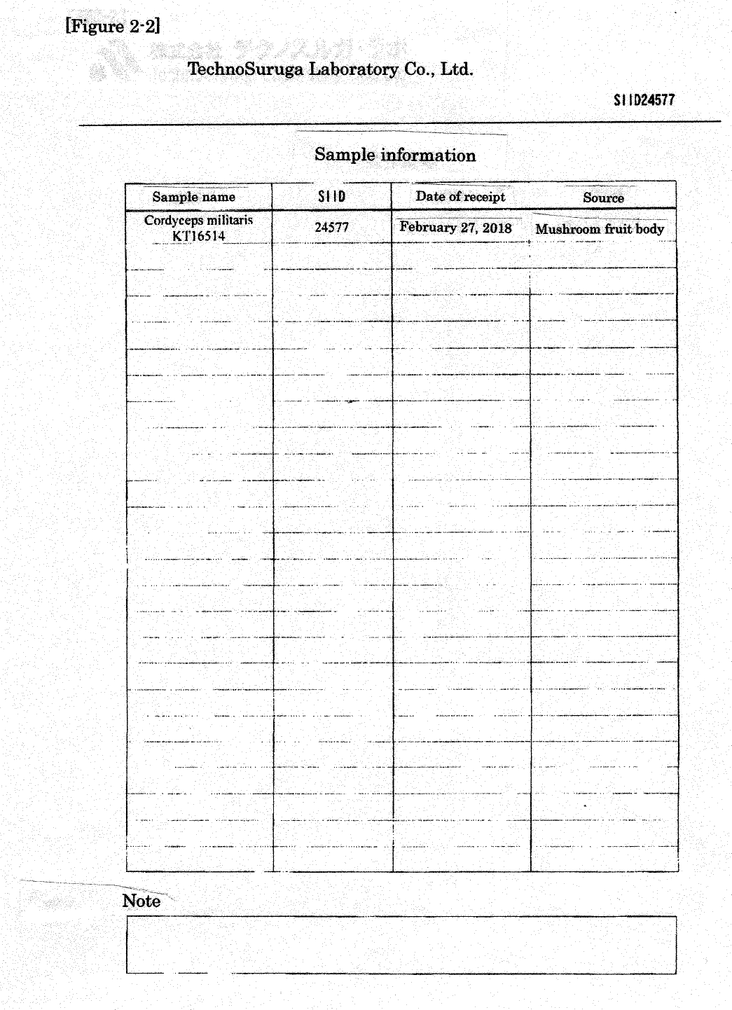

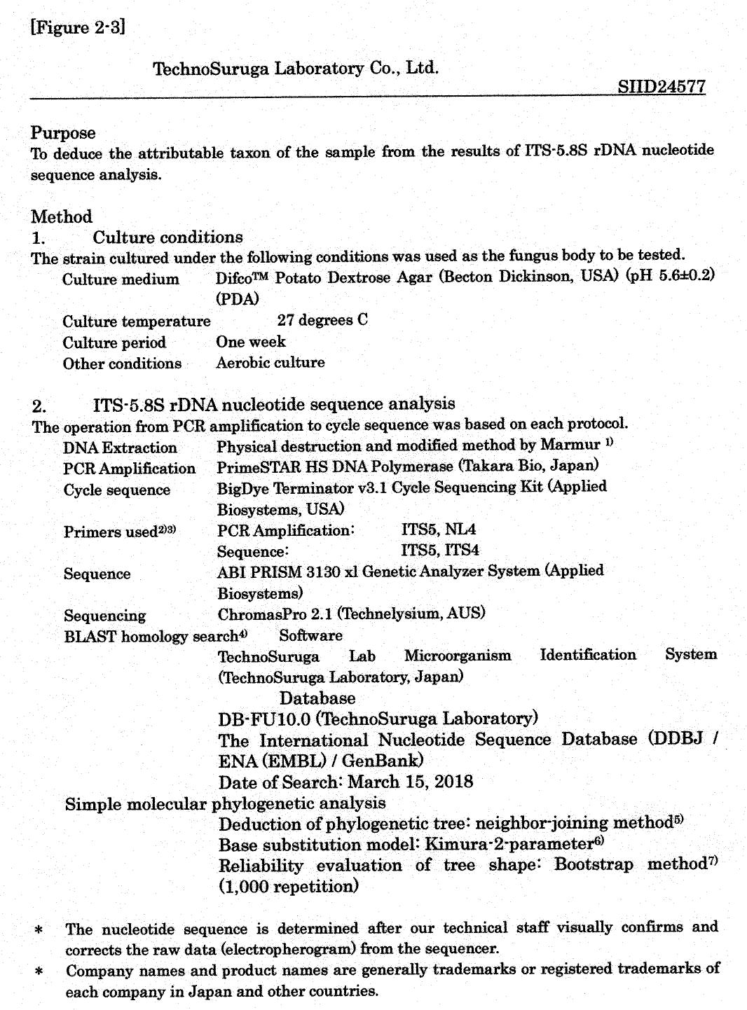

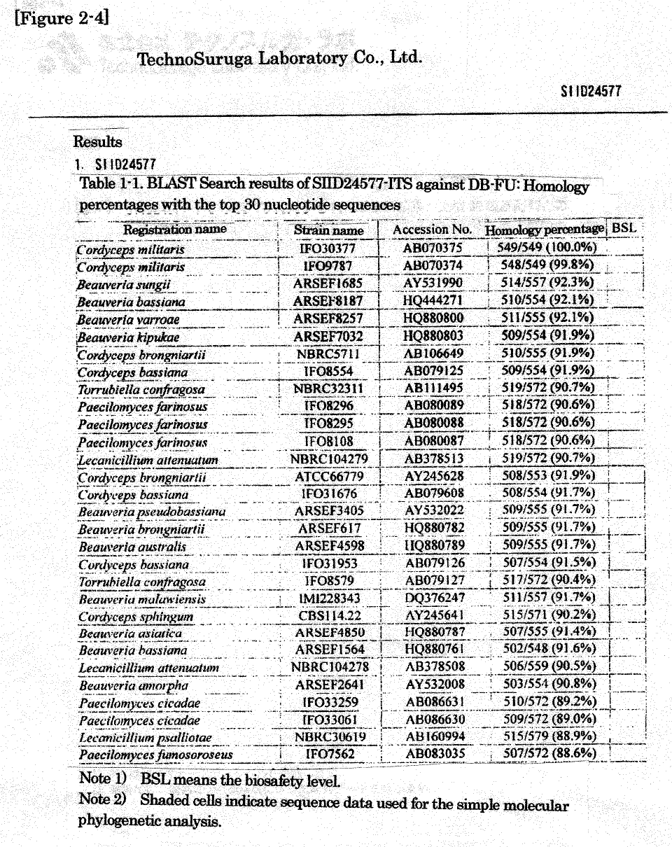

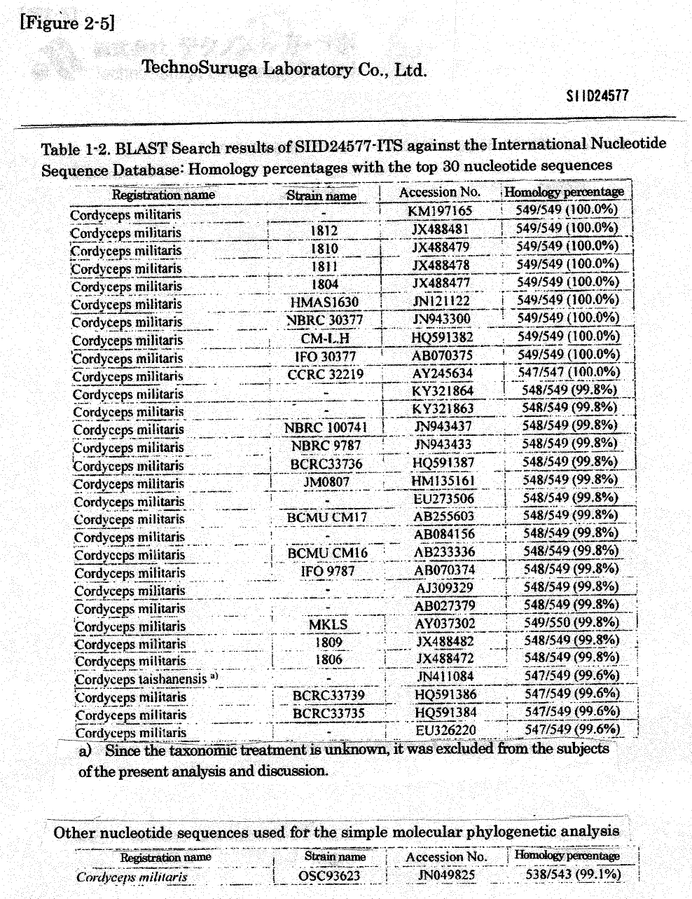

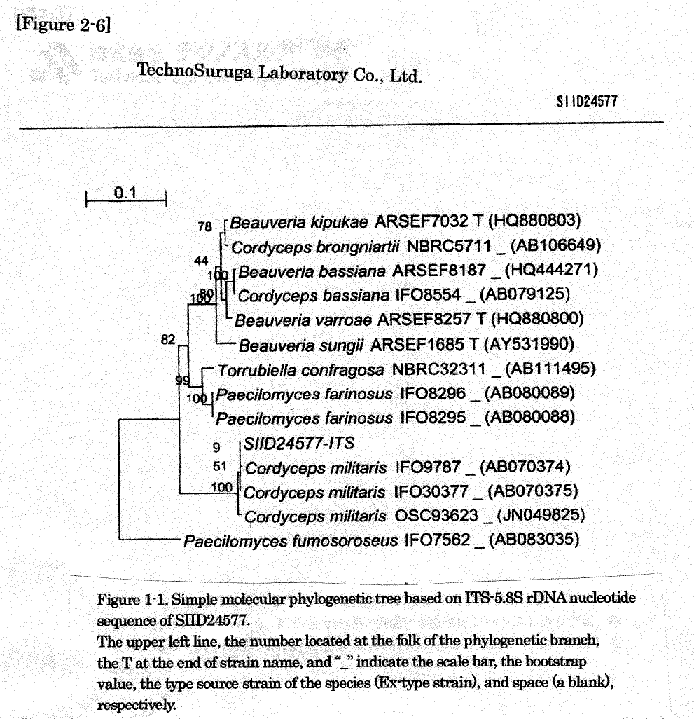

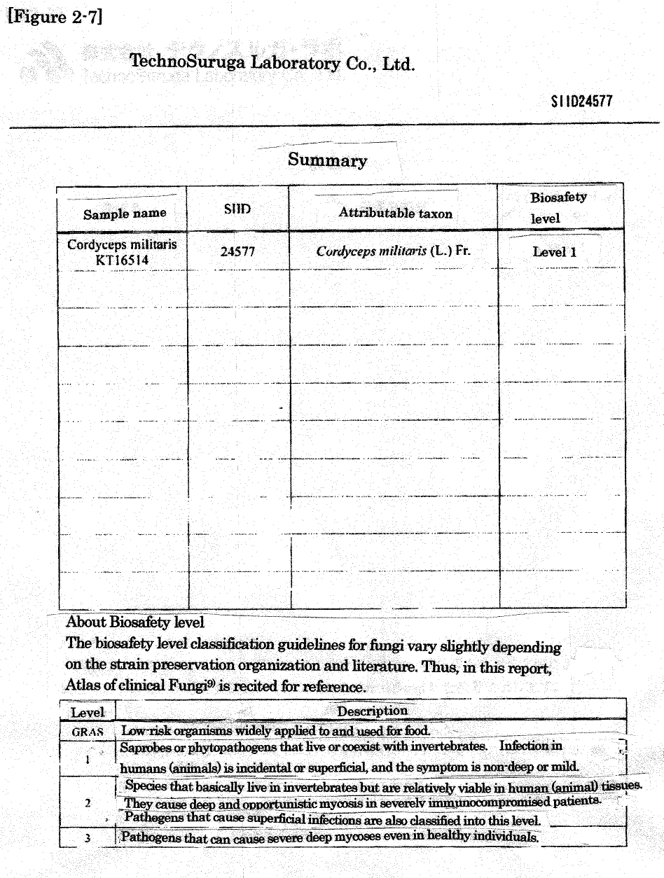

The composition of the present invention comprises culture of Cordyceps militalis in genuses Cordyceps or an extract from the culture. As Cordyceps militalis, many strains, e.g., those described in FIGS. 1-2 and 1-3 and FIGS. 2-4 and 2-5, are known, and every strain may be used. Further, as shown in FIGS. 1-1 to 1-4, Cordyceps militalis FT26K3, of which gene has a high homology rate in comparison with these publicly known strains, and as shown in FIGS. 2-1 to 2-7, Cordyceps militalis KT16514, of which gene has a high homology rate in comparison with these publicly known strains, can also be used. The present inventors gave names to these strains.

Cordyceps militalis FT26K3 is a strain, of which mycelia were purely separated successfully from a fruit body of Cordyceps militalis which grew wild in a mountain forest in Fukushima, Japan. Cordyceps militalis KT16514 is a strain, of which mycelia were purely separated successfully from a fruit body of Cordyceps militalis which grew wild in a mountain forest in Tochigi, Japan.

In the present invention, culture of mycelia or a fruit body of Cordyceps militalis is used. The strain of Cordyceps militalis is maintained by preparing a strain for storing. By preparing a slant medium in a test tube which has been sterilized by using, e.g., potato-dextrose-agar medium, malt extract-agar medium, potato-glucose-agar medium or the like, inoculating mycelial pellet to the medium and culturing, a strain for storing is prepared. The culturing conditions are, e.g., at 20 to 30 degrees C. for 2 to 6 weeks, preferably at 24 to 27 degrees C. for 3 to 5 weeks. The strain after culturing is stored at room temperature or in a refrigerator.

Generally, before large-scale culture, spawn for inoculation is prepared from the strain for storing. For culturing spawn for inoculation of mushroom, heretofore solid medium comprising sawdust or the like as a main component has been used. However, in recent years, liquid medium is also used. Examples of the liquid medium that is used for culturing spawn include SMY medium (1% by weight of sucrose, 1% by weight of a malt extract, 0.4% by weight of a yeast extract, balance of water), MY medium (0.4% by weight of glucose, 1% by weight of a malt extract, 0.4% by weight of a yeast extract, balance of water), PD medium (20% by weight of potato, 2% by weight of glucose, balance off water), M medium (2% by weight of a malt extract, balance of water), and mediums prepared by further adding other nutrient components to these mediums. Specific examples of the liquid medium which is used for culturing spawn include an aqueous solution comprising glucose, a yeast extract, rice bran (KOMENUKA), sake lees, soybean powder, sodium aspartate and water, and an aqueous solution comprising glucose, rice bran (AKANUKA), soybean powder, a yeast extract, sodium aspartate and water. The conditions for culturing spawn by using liquid medium are under stirring, e.g., 20 to 30 degrees C. for 3 to 10 days, preferably 24 to 27 degrees C. for 6 to 8 days.

The large-scale culture of culture which is used for the composition of the present invention may be performed by solid culture or liquid culture. The solid culture of mycelia can be performed by using, e.g., high protein medium comprising soybean or the like (e.g., soybean medium; it comprises soybean (may be HIKIWARI (finely crushed one) or powder), rice (may be brown rice or white rice) and water). Cordyceps militalis belongs to genus Cordyceps, and in nature grows up by using pupa of Antheraea yamamai (Japanese Oak Silkmoth) and the like as a host. Thus, solid medium (e.g., pupa medium; it comprises pupa of silkworm (may be powder), soybean (may be HIKIWARI (finely crushed one) or powder), rice (may be brown rice or white rice) and water) comprising as a protein a mite's body, e.g., pupa of silkworm (may be a powder) such as, e.g., Antheraea yamamai or white silkworm (domesticated silkworm), can also be used. In the above solid culture medium, instead of rice, barley, wheat, wheat bran or rice bran (AKANUKA) can also be used. The solid culture may be performed by inoculating spawn for inoculation to sterilized solid medium comprising a large amount (about 40 to 60% of total amount of the medium) of moisture, and culturing, e.g., at 20 to 30 degrees C. for 20 to 40 days, preferably at around 24 degrees C. for about 30 days.

Mycelia can also be cultured in a large-scale by liquid culture. Specific examples of the liquid medium which is used for large-scale culture include an aqueous solution comprising glucose, a yeast extract, rice bran (KOMENUKA), sake lees, soybean powder, sodium aspartate, an inorganic substance and water, and another aqueous solution comprising glucose, rice bran (AKANUKA), soybean powder, a yeast extract, sodium aspartate and water. The large-scale culture by using liquid medium may be performed by inoculating spawn after sterilization and cooling by an ordinary method, and culturing under aeration, e.g., at 20 to 30 degrees C. for 10 to 30 days, preferably at around 25 degrees C. for about 21 days under an aeration amount of 0.5 to 5 VVM (an aeration amount per one minute per unit volume).

After the culture medium was totally filled with the mycelia, the mycelia is transferred to solid medium for culturing a fruit body, and then the fruit body is formed by light stimulation or carbon dioxide stimulation. Specific examples of the solid medium for culturing a fruit body include one comprising pupa or pupa powder, rice bran (KOMENUKA), dried bread-like pieces derived from wheat (KOMUGIHU) and water, and having an aqueous rate of 60 to 65% by weight, and another one comprising waste obtainable after extracting coffee, chip dust, brown rice, soybean, soybean powder, rice husk and water.

After performing large-scale culture, the mycelia or the fruit body of Cordyceps militalis collected therefrom, or the solid culture or the liquid culture itself is subjected to freeze-dry or spray-dry. The dried one thus obtained can be directly used as the composition of the present invention or a part of the composition. A solvent extraction from the mycelia or the fruit body collected or from the solid culture or the liquid culture is performed, and liquid which was used for extraction is obtained by solid-liquid separation. The liquid which was used for extraction by itself, its concentrated one, or its freeze-dried or spray-dried one can be used as the composition of the present invention or a part of the composition. At need, a pH adjuster or a base for powdering, for example, can be added to the liquid which was used for extraction.

The solvent for extraction is, e.g., water or an organic solvent. When the organic solvent is used, those which can be used for a food or a beverage or a drug are preferable. Examples thereof include acetone, hexane, butanol, ethanol and the like. Among them, ethanol which has been diluted with water to be an appropriate concentration is preferable. Regarding the extraction conditions, when the extraction solvent is water, it is a hot water extraction at 95 to 100 degrees C. for 30 to 120 minutes. When the solvent is diluted ethanol, ethanol having a concentration of 20 to 50% by weight is preferably used, ethanol having a concentration of 30 to 40% by weight is more preferably used, and a combination of a warming extraction at about 50 to 65 degrees C. with a boiling extraction at about 90 to 96 degrees C. is preferable.

Because the composition of the present invention has an erythropoietin-inducing activity, it can be used as a drug for prevention or treatment of anemia such as renal anemia and the like by expecting a hematopoiesis activity of its erythrocytic series. Also, the composition of the present invention can be used in foods, e.g., foods for specified health use, foods exhibiting its function, special-use foods and supplements, with the purpose of prevention of anemia.

The embodiment of the composition of the present invention is not especially limited. When it is a food or a beverage, its embodiment is solid (including powder, granules and tablet), paste, liquid and the like. When it is a drug or a supplement, its embodiment is tablet, capsule, syrup, powdered medicine, granules, subtle granules and the like.

The composition of the present invention may contain an optional component in addition to culture of Cordyceps militalis or an extract from the culture, with the proviso that it does not give an adverse effect to the erythropoietin-inducing activity. Examples of the optional component include a sweetening agent, an agent for acidic taste, a corrective, a binder, a disintegrating agent, a lubricant, a coating agent, an excipient, a solubilizing agent and a suspending agent.

The composition of the present invention is derived from a natural product and is high in safety as hereinafter defined in Examples. Thus, it is seemed not to occur any particular problem if a large amount of it is consumed. Examples of amount of ingestion are, per one day for adult, 1 to 50 g for example, preferably 6 to 30 g, by expressing the amount of the culture contained in the composition, and 0.1 to 5.0 g for example, preferably 0.5 to 2.0 g by expressing the amount of the extract contained in the composition.

### EXAMPLES

The present invention will be specifically explained by showing examples as follows.

### [Example 1] Method for Culturing Mycelia of Cordyceps militalis

The method for culturing mycelia of Cordyceps militalis is as follows.

1. Method for Storing a Strain of Cordyceps militalis By filling to a test tube (18 mmΦ) potato-dextrose-agar medium which was prepared by an ordinary method and becoming solidified the agar, a slant was prepared. By using inoculating loop, mycelial pellet of Cordyceps militalis having a length of about 5 to 8 mm was inoculated at a center of the slant. Thereafter, culture was performed at 24 to 27 degrees C. for 3 to 5 weeks. A strain after culture was stored at room temperature or in a refrigerator at 10 degrees C.

The mycelial pellet is a strain for storing, which is derived from purely separated one from each of Cordyceps militalis FT26K3 and Cordyceps militalis KT16514 which grew wild. FIGS. 1-1 to 1-4 and FIGS. 2-1 to 2-7 show respectively that they are Cordyceps militalis.

### 2. Method for Culturing Spawn for Inoculation

A solution having the following composition, 150 mL, was poured into an Erlenmeyer flask having a content of 500 mL, and was sterilized at 121 degrees C. for 15 minutes by an ordinary method. After the temperature of the solution became 25 degrees C. or lower, mycelial pellet having a length of about 5 to 8 mm was inoculated to the solution from the strain for storing of Cordyceps militalis FT26K3. Separately, to a solution which was prepared in a same manner and which was contained in the flask, mycelial pellet having a length of about 5 to 8 mm was inoculated from the strain for storing of Cordyceps militalis KT16514. By culturing the solutions at 24 to 27 degrees C. for 7 days while stirring at 120 rpm, primary spawns for inoculation were obtained. Further, 1500 mL of a solution, which was the same as that used for culturing the primary spawn, was poured into an Erlenmeyer flask having a content of 5 L, and was sterilized by an ordinary method. After cooling, total volumes of two types of the primary spawns for inoculation were inoculated to the above solution. By culturing at 24 to 27 degrees C. for 7 to 10 days, spawn for liquid culture and solid culture was obtained.

TABLE 1
Composition of solution which was used
for culturing spawn for inoculation

| Name of | Percent |
|---|---|
| component | (% by weight) |
| Glucose | 1~5 |
| Yeast extract | 0.5~4 |
| Rice bran (KOMENUKA) | 0.5~2 |
| Sake (SEISHU) lees | 0.5~5 |
| Soybean powder | 0.1~2 |
| Sodium aspartate | 0.05~1 |
| Water | Balance |

### 3. Method for Performing Large-Scale Culture

### 3-1. Method for Performing Liquid Culture

A solution, 400 L, having the following composition was poured into a fermenter having a content of 500 L, and was sterilized by an ordinary method. After the temperature of the solution became 25 degrees C. or lower, 3 L of spawn for inoculation, which was prepared by the same method as that described in item 2., was inoculated. By culturing at 25 degrees C. for 21 days under an aeration amount of 0.5 VVM (an aeration amount per one minute per unit volume), liquid culture was obtained.

TABLE 2
Composition of medium which was used for
large-scale production by liquid culture

| Name of | Percent |
|---|---|
| component | (% by weight) |
| Glucose | 3 |
| Yeast extract | 0.5 |
| Rice bran (KOMENUKA) | 2 |
| Sake (SEISHU) lees | 2 |
| Soybean powder | 0.5 |
| Sodium aspartate | 0.1 |
| Mineral | 0.2 |
| Water | Balance |

### 3-2. Method for Performing Solid Culture

### (1) Method for Using Pupa Medium

Medium, 800 g, having the following composition was put into an incubator and was sterilized by an ordinary method. After the temperature of the medium became 25 degrees C. or lower, 10 ml of the spawn for inoculation, which was prepared by the same method as that described in item 2., was inoculated. By culturing at 24 degrees C. for 30 days, solid culture (A) was obtained.

TABLE 3
Composition of pupa medium
which was used for large-scale
production by solid culture

| | Weight |
|---|---|
| Name of component | (kg) |
| Powder of pupa of silkworm | 102 |
| Soybean powder | 34 |
| Brown rice | 20 |
| Sodium glutamate | 1 |
| Water | 113 |

### (2) Method for Using Soybean Medium

Medium, 1000 g, having the following composition was put into an incubator and was sterilized by an ordinary method. After the temperature of the medium became 25 degrees C. or lower, 10 ml of the spawn for inoculation, which was prepared by the same method as that described in item 2., was inoculated. By culturing at 24 degrees C. for 30 days, solid culture (B) was obtained.

TABLE 4
Composition of soybean medium
which was used for large-scale
production by solid culture

| | Weight |
|---|---|
| Name of component | (kg) |
| Soybean | 120 |
| Brown rice | 30 |
| Water | 170 |

### [Example 2] Method for Preparing Specimen for Administering to Mice

The specimen for administering to mice was prepared as follows.

1. Method for Preparing Extract Powder (Symbol for Mouse Administration Test: CM-A) from Cultured Mycelia of Cordyceps militalis

Seventy (70) kg of solid culture (A), 90 L of 85% ethanol, 70 L of liquid culture and 50 L of tap water were put into a vessel for extraction, and they were maintained at 55 to 58 degrees C. for 60 minutes and then 92 to 95 degrees C. for 60 minutes. After the temperature of the content of the vessel for extraction became 60 degrees C. or lower, the content was pressed and filtered. The filtrate was concentrated until its solid concentration reached to 13% by weight. To the obtained and concentrated liquid, 1 kg of citric acid was added, and 6 kg of maltodextrin was added and dissolved. Thus, 120 L of a raw feedstock for freeze dry was obtained. This raw feedstock was freeze-dried by an ordinary method, and the freeze-dried one thus obtained was crushed. Thereafter, for watervaporproof, maltodextrin powder was added. Thus, an extract powder (symbol for mouse administration test: CM-A) from cultured mycelia of Cordyceps militalis having analysis values described in the following Table was obtained.

TABLE 5
Analytical result of extract powder
(symbol for mouse administration
test: CM-A) from cultured mycelia
of Cordyceps mintalis

| Item | Result |
|---|---|
| Moisture | 3.2 g/100 g |
| Protein | 8.5 g/100 g |
| Lipid | 1.4 g/100 g |
| Ash | 1.5 g/100 g |
| Carbohydrate | 85.4 g/100 g |
| Dietary fiber | 1.5 g/100 g |
| Energy | 388 Kcal/100 g |
| Arsenic | Not detected |
| Heavy metal | Not detected |
| General plate count | 300 counts |
| | or less/g |
| Coliform bacilli | Negative |
| Staphylococcus | Negative |
| aureus | |

2. Method for Preparing Protein Powder (Symbol for Mouse Administration Test: CM-B) of Cultured Mycelia of Cordyceps militalis

By sterilizing solid culture (B) at 121 degrees C. for 20 minutes, a raw feedstock for freeze dry was obtained. This raw feedstock was freeze-dried by an ordinary method, and the freeze-dried one thus obtained was crushed. Thus, a protein powder (symbol for mouse administration test: CM-B) of cultured mycelia of Cordyceps militalis having analysis values described in the following Table was obtained.

TABLE 6
Analytical result of protein powder
(symbol for mouse administration
test: CM-B) of cultured mycelia
of Cordyceps militalis

| Item | Result |
|---|---|
| Moisture | 3.0 g/100 g |
| Protein | 37.6 g/100 g |
| Lipid | 19.8 g/100 g |
| Ash | 4.5 g/100 g |
| Carbohydrate | 35.1 g/100 g |
| Dietary fiber | 16.3 g/100 g |
| Energy | 469 Kcal/100 g |
| Arsenic | Not detected |
| Heavy metal | 9 ppm |
| General plate count | 3.5 × 10⁴ counts/g |
| Coliform bacilli | Negative |
| Staphylococcus | Negative |
| aureus | |

### [Example 3] Examination of Administrations of Extract Powder (CM-A) from Cultured Mycelia of Cordyceps militalis and Protein Powder (CM-B) of Cultured Mycelia of Cordyceps militalis to Mice (No. 1)

By administering CM-A or CM-B to mice, safeties of them were examined. And at the same time, their actions to the components in blood, erythropoietin concentration and the like were examined.

1. Examination conditions

### (1) Animals Used: ICR Male Mice

Sixty (60) of ICR male mice of 7 weekly age were purchased. After preliminary rearing of 1 week, 42 mice were used for the examination.

### (2) Administration Amount and Administration Method of Specimen

Specimen (CM-A or CM-B) was dispersed in 0.3 mL of distilled water and dissolved. It was orally administered by using stomach sound for mouse. To the control group, 0.3 mL of distilled water was orally administered in a similar way. The frequency of administration was 1 administration a day for 6 consecutive days. The administration amount of the specimen was 200 mg, 400 mg or 800 mg per 1 kg of body weight of mouse per one administration. Namely, for the administration groups shown in Table 7, the examination was performed.

TABLE 7
Structure of administration groups for
which the examination was performed

| | Amount Administered | | | |
|---|---|---|---|---|
| | 0 mg/kg- | 200 mg/kg- | 400 mg/kg- | 800 mg/kg- |
| | body weight/ | body weight/ | body weight/ | body weight/ |
| Group | once | once | once | once |
| Control group | N = 6 | | | |
| Administration | | N = 6 | N = 6 | N = 6 |
| group of CM-A | | | | |
| Administration | | N = 6 | N = 6 | N = 6 |
| group of CM-B | | | | |

### (3) Dissection of Mice and Collection of Blood Samples

At 24 hours after the last administration of the specimen, by the excess inhalation of an inhalation anesthetic for animals, “Isoful” (isoflurane; made by DS Pharma Animal Health Co., Ltd.), mice were subjected to euthanasia. Immediately after the euthanasia, collection of blood with addition of heparin was performed from aorta abdominalis. By subjecting to centrifugal separation part of the whole blood thus obtained to which heparin had been added, blood plasma to which heparin had been added was obtained. This blood plasma was frozen and stored at −30 degrees C. immediately after the separation.

### (4) Examination Method and its Results

### (4-1) Examination Regarding Safety

### (4-1-1) Variations of Body Weights of Mice (Determination of Body Weight During Examination Period)

During examination period, at fasting (2 p.m. to 4 p.m.) before administration of specimen, body weights of mice were determined. Table 8 shows the results. Regarding the control group, the body weights at day 6 increased about 3.5% by weight as compared to those before the start of the examination. Regarding the CM-A administration groups, although they were affected by the administration amount, increases of 4.0 to 5.8% by weight were observed. Regarding the CM-B administration groups, although they were affected the administration amounts, increases of 1.6 to 4.8% by weight were observed. However, regarding the body weight, every administration group did not show statistical significant difference relative to the control group through the examination period.

TABLE 8
Variations of body weights of mice to which extract from Cordyceps militalis
was administered
(Unit: g)

| | | Time determined | | | |
|---|---|---|---|---|---|
| Group | | Day 0 | Day 2 | Day 4 | Day 6 |
| CM-A | Control group | 37.2 ± 1.65 | 37.9 ± 2.18 | 38.4 ± 2.33 | 38.5 ± 2.37 |
| | Administration | 37.7 ± 1.13 | 38.7 ± 1.80 | 39.4 ± 1.94 | 39.9 ± 2.22 |
| | group of | | | | |
| | 200 mg/kg- | | | | |
| | body weight/once | | | | |
| | Administration | 37.1 ± 1.87 | 37.2 ± 1.93 | 38.0 ± 2.36 | 38.9 ± 2.52 |
| | group of | | | | |
| | 400 mg/kg- | | | | |
| | body weight/once | | | | |
| | Administration | 37.5 ± 2.18 | 37.9 ± 2.49 | 38.6 ± 2.25 | 39.0 ± 2.09 |
| | group of | | | | |
| | 800mg/kg- | | | | |
| | body weight/once | | | | |
| CM-B | Control group | 37.2 ± 1.65 | 37.9 ± 2.18 | 38.4 ± 2.33 | 38.5 ± 2.37 |
| | Administration | 37.3 ± 2.81 | 38.2 ± 3.40 | 38.5 ± 3.94 | 39.1 ± 3.27 |
| | group of | | | | |
| | 200 mg/kg- | | | | |
| | body weight/once | | | | |
| | Administration | 37.3 ± 0.76 | 37.5 ± 1.06 | 38.2 ± 1.54 | 38.3 ± 1.23 |
| | group of | | | | |
| | 400 mg/kg- | | | | |
| | body weight/once | | | | |
| | Administration | 36.8 ± 0.69 | 36.7 ± 0.90 | 37.4 ± 1.13 | 37.4 ± 1.06 |
| | group of | | | | |
| | 800 mg/kg- | | | | |
| | body weight/once | | | | |

### (4-1-2) Observances of Mice in Motility and Appetite During Examination Period, and Examination of Appearances of Mice Before Dissection

During examination period, motility and appetite in mice were observed. However, there were no abnormity in all mice. After euthanasia, appearances of mice were examined. However, there were no abnormities such as blot of body hair, diarrhea and the like in all mice.

### (4-1-3) Visual Examinations of Organs During Dissection

After euthanasia, celiotomy was performed and regarding each organ, position, configuration, existence or not of bleeding, and existence or not of adhesion were visually examined. However, regarding these components of the examinations, there were no abnormities in all mice.

### (4-1-4) Determination of Body Weights Immediately Before Dissection and Determination of Organs' Weights at Dissection

At immediately before dissection, body weights of mice were determined. Also, weights of liver, kidney, spleen, thymus gland and testicle which had been extirpated were respectively determined. Table 9 shows the results.

Body weight: Every administration group did not show statistical significant difference relative to the control group.

Liver weight: Every administration group did not show statistical significant difference relative to the control group.

Kidney weight: When averages of groups were compared to each other, every administration group was heavier than the control group. However, there were large individual differences, and only the administration group of CM-A 200 mg/kg-body weight/once (level of significance: less than 1%) and the administration group of CM-A 800 mg/kg-body weight/once (level of significance: less than 5%) were determined as to be significantly heavy relative to the control group.

Spleen weight: Relative to the control group, there was a group in which its weight increased and there was a group in which its weight decreased. Namely, there was no specific tendency. Regarding the administration group of CM-B 800 mg/kg-body weight/once, its weight increased with level of significance of less than 5%.

Thymus gland weight: Relative to the control group, there was a group in which its weight increased and there was a group in which its weight decreased. Namely, there was no specific tendency and also there was no administration group having statistically significant difference.

Testicle weight: Relative to the control group, there was a group in which its weight increased and there was a group in which its weight decreased. Namely, there was no specific tendency and also there was no administration group having statistically significant difference.

From the above, it can be said that in both of CM-A administration groups and CM-B administration groups, the kidney weights had a tendency to increase, and that there was no abnormity by which safety is concerned.

TABLE 9
Body weights of mice to which extract from Cordyceps militalis was administered immediately before dissection, and weights of viscera

| | | Object to be determined | | | | | |
|---|---|---|---|---|---|---|---|
| | | Body weight | | | | | |
| | | immediately | | | | Thymus | |
| | | before | | | | gland | Testicle |
| Group | | dissection (g) | Lever (mg) | Kidney (mg) | Spleen (mg) | (mg) | (mg) |
| CM-A | Control group | 38.4 ± 2.3 | 2044 ± 267.6 | 609 ± 67.9 | 133 ± 42.2 | 54 ± 10.6 | 253 ± 23.5 |
| | Administration group of | 39.7 ± 2.2 | 2177 ± 281.8 | 716 ± 31.0* | 160 ± 49.3 | 41 ± 18.3 | 240 ± 6.5 |
| | 200 mg/kg-body weight/once | | | | | | |
| | Administration group of | 38.6 ± 2.4 | 2037 ± 306.3 | 646 ± 67.6 | 137 ± 28.1 | 58 ± 15.2 | 246 ± 30.7 |
| | 400 mg/kg-body weight/once | | | | | | |
| | Administration group of | 38.6 ± 2.3 | 2050 ± 152.0 | 659 ± 43.1** | 121 ± 13.2 | 56 ± 7.8 | 250 ± 40.9 |
| | 800 mg/kg-body weight/once | | | | | | |
| CM-B | Control group | 38.4 ± 2.3 | 2044 ± 267.6 | 609 ± 67.9 | 133 ± 42.2 | 54 ± 10.6 | 253 ± 23.5 |
| | Administration group of | 38.9 ± 3.2 | 2128 ± 274.1 | 715 ± 123.9 | 126 ± 37.5 | 54 ± 17.7 | 228 ± 23.2 |
| | 200 mg/kg-body weight/once | | | | | | |
| | Administration group of | 38.0 ± 1.3 | 2121 ± 125.1 | 687 ± 74.9 | 148 ± 47.1 | 48 ± 12.3 | 264 ± 20.0 |
| | 400 mg/kg-body weight/once | | | | | | |
| | Administration group of | 37.0 ± 1.3 | 2036 ± 119.0 | 638 ± 128.7 | 273 ± 117.4** | 42 ± 6.0 | 247 ± 36.6 |
| | 800 mg/kg-body weight/once | | | | | | |

*There is a significant difference relative to the control group with level of significance of less than 1%.
**There is a significant difference relative to the control group with level of significance of less than 5%.

### (4-2) Examination of Blood

By using whole blood which was collected immediately after the euthanasia, to which heparin had been added, number of leucocyte, number of erythrocyte, hemoglobin concentration, hematocrit value and number of thrombocyte were determined with an automatic equipment for determining hematocytes (made by Sysmex Corporation, pocH-1000. Table 10 shows the results.

Number of leucocyte: When averages of the groups were compared to each other, every administration group was superior to the control group. The groups which were judged as to be significantly superior to the control group were the administration group of CM-A 200 mg/kg-body weight/once (level of significance: less than 5%), and the administration group of CM-B 200 mg/kg-body weight/once (level of significance: less than 1%), the administration group of CM-B 400 mg/kg-body weight/once (level of significance: less than 5%) and the administration group of CM-B 800 mg/kg-body weight/once (level of significance: less than 5%).

Number of erythrocyte: Relative to the control group, there was a group in which number of erythrocyte increased and there was a group in which number of erythrocyte decreased. Namely, there was no specific tendency. Regarding the administration group of CM-A 800 mg/kg-body weight/once, number of erythrocyte increased with level of significance of less than 5%.

Hemoglobin concentration: There was a tendency that hemoglobin concentration was comparable to or slightly higher than the control group. Regarding the administration group of CM-A 800 mg/kg-body weight/once, hemoglobin concentration increased with level of significance of less than 5%.

Hematocrit value: Relative to the control group, there was a group in which hematocrit value increased and there was a group in which hematocrit value decreased. Namely, there was no specific tendency. Regarding the administration group of CM-A 800 mg/kg-body weight/once, hematocrit value increased with level of significance of less than 1%.

Number of thrombocyte: The CM-A administration groups had a tendency that numbers of thrombocyte were comparable to or decreased somewhat than the control group. On the other hand, the CM-B administration groups had a tendency that numbers of thrombocyte increased somewhat relative to the control group. Especially, in the administration group of CM-B 800 mg/kg-body weight/once, number of thrombocyte increased with level of significance of less than 5%.

From the above, it can be said that both of CM-A and CM-B had a tendency to increase the number of leucocyte when they were orally ingested,

TABLE 10
Examination results of bloods which were collected immediately after dissection
from mice to which extract from Cordyceps militalis had been administered

| | | Object to be determined | | | | |
|---|---|---|---|---|---|---|
| | | Number of | Number of | Hemoglobin | Hematocrit | Number of |
| | | leucocyte | erythrocyte | concentration | value | thrombocyte |
| Group | | (cells/μl) | (cells × 10⁴/μl) | (g/dl) | (%) | (cells × 10⁴/μl) |
| CM-A | Control group | 6100 ± 1018 | 920.2 ± 27.1 | 14.2 ± 0.4 | 52.8 ± 1.5 | 109.9 ± 19.8 |
| | Administration group of | 7883 ± 1209** | 884.5 ± 58.4 | 14.5 ± 1.1 | 51.4 ± 3.3 | 93.7 ± 17.0 |
| | 200 mg/kg-body weight/once | | | | | |
| | Administration group of | 6700 ± 1208 | 888.3 ± 46.8 | 14.2 ± 0.6 | 51.9 ± 3.2 | 103.9 ± 15.7 |
| | 400 mg/kg-body weight/once | | | | | |
| | Administration group of | 7350 ± 2599 | 969.7 ± 38.4** | 15.1 ± 0.6** | 56.0 ± 1.7* | 109.5 ± 26.7 |
| | 800 mg/kg-body weight/once | | | | | |
| CM-B | Control group | 6100 ± 1018 | 920.2 ± 27.1 | 14.2 ± 0.4 | 52.8 ± 1.5 | 109.9 ± 19.8 |
| | Administration group of | 8717 ± 1861** | 915.3 ± 54.0 | 14.4 ± 0.8 | 52.7 ± 3.1 | 120.1 ± 19.5 |
| | 200 mg/kg-body weight/once | | | | | |
| | Administration group of | 8150 ± 1190* | 942.0 ± 61.3 | 14.7 ± 0.9 | 53.9 ± 3.6 | 120.5 ± 21.5 |
| | 400 mg/kg-body weight/once | | | | | |
| | Administration group of | 8133 ± 1851** | 902.7 ± 63.3 | 14.2 ± 0.6 | 52.0 ± 3.3 | 148.5 ± 30.4** |
| | 800 mg/kg-body weight/once | | | | | |

*There is a significant, difference relative to the control group with level of significance of less than 1%.
**There is a significant difference relative to the control group with level of significance of less than 5%.

### (4-3) Determination of Erythropoietin Concentration in Blood Plasma

By using Mouse Erythropoietin Quantikine ELISA Kit (R & D Systems, Cat. No. ME P00B, Lot. No. P144663), erythropoietin concentration was determined for the mouse blood plasma which was obtained by collecting at dissection with addition of heparin and separating. Table 11 and FIGS. 3 and 4 show the results.

Every administration group had a high erythropoietin concentration relative to the control group. Among them, each of the administration group of CM-A 200 mg/kg-body weight/once and the administration group of CM-B 800 mg/kg-body weight/once showed a significantly high erythropoietin concentration relative to the control group with level of significance of less than 1%.

From the above, it was understood that there was a tendency to increase the erythropoietin concentration when CM-A or CM-B was orally ingested. For confirmation, the erythropoietin concentration of each of CM-A and CM-B was determined. However, erythropoietin was not detected.

TABLE 11
Erythropoietin concentrations of blood plasmas which were
collected immediately after dissection from mice to which
extract from Cordyceps militalis had been administered
(Unit: pg/ml)

| | | Erythropoietin |
|---|---|---|
| Group | | concentration |
| CM-A | Control group | 81.7 ± 18.8 |
| | Administration group of 200 | 177.4 ± 41.6* |
| | mg/kg-body weight/once | |
| | Administration group of 400 | 94.7 ± 19.2 |
| | mg/kg-body weight/once | |
| | Administration group of 800 | 93.9 ± 35.8 |
| | mg/kg-body weight/once | |
| CM-B | Control group | 81.7 ± 18.8 |
| | Administration group of 200 | 86.4 ± 32.7 |
| | mg/kg-body weight/once | |
| | Administration group of 400 | 150.8 ± 78.3 |
| | mg/kg-body weight/once | |
| | Administration group of 800 | 229.3 ± 25.2* |
| | mg/kg-body weight/once | |

*There is a significant difference relative to the control group with level of significance of less than 1%.
**There is a significant difference relative to the control group with level of significance of less than 5%.

### (4-4) Histological Examination of Mouse's Kidney

Kidney's tissue specimens (4 specimens for each administration group) of administration groups of CM-A (0 mg/kg-body weight/once (control), 200 mg/kg-body weight/once, 400 mg/kg-body weight/once and 800 mg/kg-body weight/once), in which kidney weights significantly increased, were prepared, and hypertrophy of the kidney was histologically examined. Pathological histological examinations of mice's kidneys were asked to New Histo. Science Laboratory Co., Ltd. Kidney's tissue specimens that had been prepared by HE staining were examined under the microscope.

Regarding each of the administration group of 400 mg/kg-body weight/once and the administration group of 800 mg/kg-body weight/once, 1 specimen among 4 specimens showed microscopic vacuolar degeneration at proximal uriniferous tubule. The vacuolar degeneration may be a cause that the administration group of 400 mg/kg-body weight/once and the administration group of 800 mg/kg-body weight/once did not show high erythropoietin concentrations relative to the administration group of 200 mg/kg-body weight/once. Therefore, the inventors thought that it was necessary to perform an additional test regarding variation of the erythropoietin concentration when lower amounts of CM-A were administered.

### [Example 4] Examination of Administrations of CM-A to Mice (No. 2)

By administering CM-A to mice, safety of it was examined. And at the same time, its actions to the components in blood, erythropoietin concentration and the like were examined.

### 1. Examination Conditions

### (1) Animals Used: ICR Male Mice

Thirty six (36) of ICR male mice of 8 weekly age were purchased. After preliminary rearing of 3 days, the mice were used for the examination.

### (2) Administration Amount and Administration Method of Specimen

Specimen (CM-A) was dispersed in 0.2 mL of distilled water and dissolved. It was orally administered by using stomach sound for mouse. To the control group, 0.2 mL of distilled water was orally administered in a similar way. The frequency of administration was 1 administration a day for 6 consecutive days. However, for only the administration group of 200 mg/kg-body weight/once, an administration method of 1 administration a day for 3 consecutive days was also performed in addition to 6 days administration. The administration amount of the specimen was 25 mg, 50 mg, 100 mg or 200 mg per 1 kg of body weight of mouse per 1 administration. Namely, for the administration groups shown in Table 12, the examination was performed.

TABLE 12

| | Amount administered | | | | |
|---|---|---|---|---|---|
| | 0 mg/kg- | 25 mg/kg- | 50 g/kg- | 100 mg/kg- | 200 mg/kg- |
| | body weight/ | body weight/ | body weight/ | body weight/ | body weight/ |
| Group | once | once | once | once | once |
| Control group | N = 6 | | | | |
| Administration group | | N = 6 | N = 6 | N = 6 | N = 6 |
| of CM-A (6 times) | | | | | |
| Administration group | | | | | N = 6 |
| of CM-A (3 times) | | | | | |

### (3) Dissection of Mice and Collection of Blood Samples

At 24 hours after the last administration of the specimen, by the excess inhalation of the inhalation anesthetic for animals, “Isoful” (isoflurane; made by DS Pharma-Animal Health Co., Ltd.), mice were subjected to euthanasia. Immediately after the euthanasia, collection of blood with addition of heparin was performed from aorta abdominalis. By subjecting to centrifugal separation part of the whole blood thus obtained to which heparin had been added, blood plasma to which heparin had been added was obtained. This blood plasma was frozen and stored at −30 degrees C. immediately after the separation.

### (4) Examination Method and its Results

Regarding the examination methods, descriptions of methods which are the same as those described in Example 3 are omitted and only the results are described. Different methods are described in what follows.

### (4-1) Examination Regarding Safety

### (4-1-1) Variations of Body Weights of Mice (Determination of Body Weight During Examination Period)

Table 13 shows the results.

Regarding the control group, the body weights at day 6 increased about 5.3% by weight as compared to those before the start of the examination. Regarding the CM-A administration groups, although they were affected by the administration amounts, increases of 3.9 to 5.6% by weight were observed. However, regarding the body weight, every administration group did not show statistical significant difference relative to the control group through the examination period.

TABLE 13
Variations of body weights of mice to which extract from Cordyceps militalis was administered
(Unit: g)

| | Time determined | | | |
|---|---|---|---|---|
| Group | Day 0 | Day 2 | Day 4 | Day 6 |
| Control group | 37.35 ± 0.96 | 38.90 ± 0.96 | 38.75 ± 0.72 | 39.37 ± 1.03 |
| Administration group of 25 | 36.73 ± 0.80 | 37.78 ± 0.84 | 37.40 ± 0.84 | 38.40 ± 0.78 |
| mg/kg-body weight/once | | | | |
| Administration group of 50 | 37.22 ± 1.05 | 38.38 ± 1.30 | 38.65 ± 1.20 | 38.77 ± 1.30 |
| mg/kg-body weight/once | | | | |
| Administration group of 100 | 37.23 ± 1.43 | 38.43 ± 1.53 | 38.60 ± 1.40 | 39.27 ± 1.71 |
| mg/kg-body weight/once | | | | |
| Administration group of 200 | 38.30 ± 0.75 | 39.47 ± 0.87 | 40.13 ± 0.66 | 39.75 ± 1.15 |
| mg/kg-body weight/once | | | | |

### (4-1-2) Observances of Mice in Motility and Appetite During Examination Period, and Examination of Appearances of Mice Before Dissection

During examination period, regarding the motility and the appetite in mice, there were no abnormities in all mice. Also regarding the examination of appearances of mice after euthanasia, there were no abnormities such as blot of body hair, diarrhea and the like in all mice.

### (4-1-3) Visual Examinations of Organs During Dissection

There were no abnormities in all mice in visual examinations about position, configuration, existence or not of bleeding, and existence or not of adhesion of each organ.

### (4-1-4) Determination of Body Weight Immediately Before Dissection and Determinations of Organs' Weights at Dissection

As the organs' weights at dissection, in addition to liver weight, kidney weight, spleen weight, thymus gland weight and testicle weight, the weight of fat around the testicle (intraperitoneal) was also determined. This determination was also performed for the group to which CM-A was administered in an amount of 200 mg/kg-body weight/once for 3 times (3 consecutive days). Tables 14 and 15 show the results. Table 14 shows the absolute weights of organs determined, and Table 15 shows relative weights of organs per 10 g of body weight.

Body weight: Regarding absolute weights and relative weights, every administration group did not show statistical significant difference relative to the control group.

Liver weight: Regarding absolute weights and relative weights, every administration group was heavier than the control group. Among them, regarding the absolute weights, the administration group of 25 mg/kg-body weight/once (level of significance: less than 1%) was significantly heavy relative to the control group, and regarding relative weights, the administration group of 25 mg/kg-body weight/once (level of significance: less than 5%) and the administration group of 50 mg/kg-body weight/once (level of significance: less than 5%) were significantly heavy relative to the control group.

Kidney weight: Every administration group, other than the administration group of 25 mg/kg-body weight/once, was heavier than the control group. However, the group which was judged as to be significantly heavy relative to the control group was only the relative weight (level of significance: less than 5%) of the administration group of 200 mg/kg-body weight/once (3 times).

Spleen weight: Regarding the administration group of 200 mg/kg-body weight/once (6 times), not only the absolute weight but also the relative weight significantly increased relative to the control group. Regarding the groups other than above group, the weights were almost the same as those of the control group.

Thymus gland weight: Relative to the control group, there was a group in which its weight increased and there was a group in which its weight decreased. Namely, there was no specific tendency and there was no administration group having statistically significant difference.

Testicle weight: In every 6 times administration group, the absolute weight and the relative weight significantly decreased relative to the control group. Regarding the administration group of 200 mg/kg-body weight/once (3 times), the absolute weight and the relative weight were almost the same as those of the control group.

Weight of fat around the testicle: There was a tendency that for every 6 times administration group, the absolute weight and the relative weight increased relative to the control group. However, there was no administration group having statistically significant difference relative to the control group. Regarding the administration group of 200 mg/kg-body weight/once (3 times), increase amounts of the absolute weight and the relative weight were the largest. However, there was no significant difference between it and the control group.

From the above, there were tendencies that the liver weight increased and that the thymus gland weight decreased when CM-A was orally administered. However, it can be said that there was no abnormality by which safety is concerned.

TABLE 14
Body weights immediately before dissection of mice to which extract from Cordyceps militalis was administered and absolute weights of viscera

| | Object to be determined | | | | | | |
|---|---|---|---|---|---|---|---|
| | Body weight | | | | | | |
| | immediately | | | | Thymus | | Fat around |
| | before | Liver | Kidney | Spleen | gland | Testicle | testicle |
| Group | dissection (g) | (mg) | (mg) | (mg) | (mg) | (mg) | (mg) |
| Control group | 39.1 ± 1.1 | 2039 ± 176.6 | 643 ± 49.9 | 125 ± 13.0 | 66 ± 7.8 | 239 ± 16.6 | 739 ± 148.6 |
| Administration group of | 38.2 ± 0.8 | 2253 ± 150.6* | 625 ± 42.8 | 119 ± 14.1 | 43 ± 9.4* | 246 ± 32.8 | 751 ± 150.7 |
| 25 mg/kg-body weight/once | | | | | | | |
| (6 times) | | | | | | | |
| Administration group of | 38.6 ± 1.2 | 2257 ± 163.1 | 663 ± 48.1 | 120 ± 12.5 | 49 ± 11.9** | 241 ± 34.8 | 759 ± 139.8 |
| 50 mg/kg-body weight/once | | | | | | | |
| (6 times) | | | | | | | |
| Administration group of | 38.9 ± 1.8 | 2220 ± 232.8 | 685 ± 45.0 | 130 ± 28.9 | 54 ± 8.8** | 236 ± 25.1 | 776 ± 163.3 |
| 100 mg/kg-body weight/once | | | | | | | |
| (6 times) | | | | | | | |
| Administration group of | 39.3 ± 0.9 | 2157 ± 158.4 | 692 ± 31.5 | 149 ± 21.5** | 44 ± 9.0* | 260 ± 44.4 | 856 ± 174.3 |
| 200 mg/kg-body weight/once | | | | | | | |
| (6 times) | | | | | | | |
| Administration group of | 39.1 ± 1.6 | 2174 ± 215.8 | 721 ± 79.5 | 131 ± 29.1 | 63 ± 11.8 | 238 ± 24.1 | 694 ± 138.0 |
| 200 mg/kg-body weight/once | | | | | | | |
| (3 times) | | | | | | | |

*There is a significant difference relative to the control group with level of significance of less than 1%.
**There is a significant difference relative to the control group with level of significance of less than 5%.

TABLE 15
Body weights immediately before dissection of mice to which extract from Cordyceps militalis was administered and relative weights of viscera
(per 10 g of body weight)

| | Object to be determined | | | | | | |
|---|---|---|---|---|---|---|---|
| | Body weight | | | | | | |
| | immediately | | | | Thymus | | Fat around |
| | before | Liver | Kidney | Spleen | gland | Testicle | testicle |
| Group | dissection (g) | (mg) | mg) | (mg) | (mg) | (mg) | (mg) |
| Control group | 39.1 ± 1.1 | 522 ± 45 | 164.8 ± 14.6 | 32.0 ± 2.8 | 16.8 ± 2.0 | 61.2 ± 5.5 | 189.4 ± 39.0 |
| Administration group of | 38.2 ± 0.8 | 590 ± 30** | 163.9 ± 13.6 | 31.2 ± 3.6 | 11.2 ± 2.6* | 64.4 ± 8.7 | 196.5 ± 38.2 |
| 25 mg/kg-body weight/once | | | | | | | |
| (6 times) | | | | | | | |
| Administration group of | 38.6 ± 1.2 | 585 ± 33** | 172.0 ± 13.2 | 31.0 ± 2.5 | 12.7 ± 2.9** | 62.5 ± 9.2 | 197.6 ± 41.9 |
| 50 mg/kg-body weight/once | | | | | | | |
| (6 times) | | | | | | | |
| Administration group of | 38.9 ± 1.8 | 570 ± 51 | 176.4 ± 14.9 | 33.4 ± 6.8 | 13.8 ± 2.1** | 60.9 ± 7.7 | 198.6 ± 37.1 |
| 100 mg/kg-body weight/once | | | | | | | |
| (6 times) | | | | | | | |
| Administration group of | 39.3 ± 0.9 | 548 ± 29 | 176.1 ± 6.9 | 37.9 ± 5.2** | 11.3 ± 2.4* | 66.0 ± 9.9 | 217.1 ± 39.7 |
| 200 mg/kg-body weight/once | | | | | | | |
| (6 times) | | | | | | | |
| Administration group of | 39.1 ± 1.6 | 555 ± 36 | 184.2 ± 15.3** | 33.4 ± 6.8 | 16.2 ± 2.9 | 60.7 ± 5.0 | 177.6 ± 34.8 |
| 200 mg/kg-body weight/once | | | | | | | |
| (3 times) | | | | | | | |

*There is a significant. difference relative to the control group with level of significance of less than 1%.
**There is a significant difference relative to the control group with level of significance of less than 5%.

### (4-2) Examination of Blood

Table 16 shows the results.

Number of leucocyte: When averages of the groups were compared to each other, every administration group was superior to the control group. The groups which were judged as to be significantly superior to the control group were 6 times administration groups, i.e., the administration group of 25 mg/kg-body weight/once (level of significance: less than 5%), the administration group of 100 mg/kg-body weight/once (level of significance: less than 5%) and the administration group of 200 mg/kg-body weight/once (level of significance: less than 5 W.

Number of erythrocyte: When averages of the groups were compared to each other, every administration group was superior to the control group. However, the group which was judged as to be significantly superior to the control group was only the administration group of 100 mg/kg-body weight/once (6 times).

Hemoglobin concentration: When averages of the groups were compared to each other, regarding the hemoglobin concentration, every administration group in the 6 times administration groups showed somewhat higher value than the control group. The administration group of 200 mg/kg-body weight/once (3 times) showed somewhat lower value than the control group. However, every group had no significant difference relative to the control group.

Hematocrit value: Relative to the control group, there was a group in which the hematocrit value increased and there was a group in which the hematocrit value decreased. Namely, there was no specific tendency. Regarding the administration group of 100 mg/kg-body weight/once (6 times), hematocrit value increased with level of significance of less than 5%.

Number of thrombocyte: Relative to the control group, there was a group in which the hematocrit value increased and there was a group in which the hematocrit value decreased. Namely, there was no specific tendency.

From the above, it can be said that CM-A had a tendency to increase the number of leucocyte when it was orally ingested,

TABLE 16
Examination results of bloods which were collected immediately after dissection
from mice to which extract from Cordyceps militalis was administered

| | Object to be determined | | | | |
|---|---|---|---|---|---|
| | | | | | Number |
| | Number of | Number of | Hemoglobin | Hematocrit | of |
| | leucocyte | erythrocyte | concentration | value | thrombocyte |
| Group | (cells/μl) | (cells × 10⁴/μl) | (g/dl) | (%) | (cells × 10⁴/μl) |
| Control group | 5333 ± 1880 | 917 ± 44.4 | 14.5 ± 0.9 | 54.1 ± 2.3 | 112.1 ± 16.3 |
| Administration group of 25 mg/kg- | 10433 ± 3968** | 927 ± 32.3 | 14.9 ± 0.7 | 53.4 ± 2.5 | 94.4 ± 20.3 |
| body weight/once (6 times) | | | | | |
| Administration group of 5 mg/kg- | 8950 ± 3980 | 945 ± 40.5 | 15.1 ± 0.6 | 55.4 ± 2.7 | 103.9 ± 28.1 |
| body weight/once (6 times) | | | | | |
| Administration group of 100 mg/kg- | 8017 ± 2113** | 977 ± 16.6** | 15.3 ± 0.3 | 57.1 ± 0.8** | 121.4 ± 23.7 |
| body weight/once (6 times) | | | | | |
| Administration group of 200 mg/kg- | 8083 ± 2069** | 943 ± 39.6 | 15.0 ± 0.7 | 55.7 ± 2.9 | 101.1 ± 40.1 |
| body weight/once (6 times) | | | | | |
| Administration group of 200 mg/kg- | 7383 ± 2363 | 857 ± 64.0 | 13.9 ± 1.2 | 50.5 ± 3.3 | 95.0 ± 29.0 |
| body weight/once (3 times) | | | | | |

*There is a significant difference relative to the control group with level of significance of less than 1%.
**There is a significant difference relative to the control group with level of significance of less than 5%.

### (4-3) Determination of Erythropoietin Concentration in Blood Plasma

Table 17 and FIG. 5 show the results.

In the administration group of 25 mg/kg-body weight/once, the administration group of 50 mg/kg-body weight/once and the administration group of 100 mg/kg-body weight/once among 6 times administration groups, and in the administration group of 200 mg/kg-body weight/once (3 times), erythropoietin concentrations that were higher than that of the control group were shown. The administration group of 200 mg/kg-body weight/once (6 times) had an erythropoietin concentration which was almost the same as that of the control group. Groups which showed significantly higher erythropoietin concentrations than the control group were only the administration group of 25 mg/kg-body weight/once (6 times) and the administration group of 200 mg/kg-body weight/once (3 times).

From the above, it was understood that there was a tendency for the erythropoietin concentration to increase when CM-A was orally ingested. For confirmation, the erythropoietin concentration of CM-A was determined. However, erythropoietin was not detected.

TABLE 17
Erythropoietin concentrations of blood plasmas which were
collected immediately after dissection from mice to which
extract from Cordyceps militalis had been administered
(Unit: pg/ml)

| | Erythropoietin |
|---|---|
| Group | concentration |
| Control group | 75.1 ± 8.10 |
| Administration group of 25 mg/kg- | 109.5 ± 31.68** |
| body weight/once (6 times) | |
| Administration group of 50 mg/kg- | 94.4 ± 27.36 |
| body weight/once (6 times) | |
| Administration group of 100 mg/kg- | 79.2 ± 19.56 |
| body weight/once (6 times) | |
| Administration group of 200 mg/kg- | 74.2 ± 8.55 |
| body weight/once (6 times) | |
| Administration group of 200 mg/kg- | 98.5 ± 22.10** |
| body weight/once (3 times) | |

*There is a significant difference relative to the control group with level of significance of less than 1%.
**There is a significant difference relative to the control group with level of significance of less than 5%.

### [Example 5] Examination of Administration of CM-A to Human

By administering CM-A to human, safety of it was examined. And at the same time, its actions to components in blood, erythropoietin concentration and the like were examined.

### 1. Examination Method

### 1-1. Ethical Practice of Examination

This examination was evaluated by the ethical committee of Japan Society of Vascular Medicine and Rheology in items of ethical and scientific acceptability and was authorized (Jun. 3, 2018; Authorization number 20180603-1). Thereafter, the inventors performed the obtaining of the subjects' agreements and overall of the examination according to the spirit of Helsinki Declaration.

### 1-2. Explanation to Subjects and Obtaining of Agreements

Before performing this examination, a written document describing the agreement of participating in the examination was handed on each subject, and the examination contents and the like were sufficiently explained. After confirming that the subject oneself understood the examination contents, the inventors received the voluntary agreement of the subject oneself in a written form.

### 1-3. Selection of Subjects Who Participate in Examination and Factors for Exclusion

### (Selection Criteria)

A person who fulfills the following items (a) and (b)

(a) An adult of a man or a woman
(b) A person who can participate in an examination in which the person continuously ingests the food to be examined for 4 months with blood drawing, and has intention of participation

### (Exclusion Criteria)

Person who corresponds to any of the following items can not be a subject of this examination.

(c) A person now experiencing any disease which must be treated, including anemia (The result of the examination of the blood that was collected before ingestion of the food to be examined is considered.)
(d) A person getting medication (including iron preparation) by a doctor during the period of ingestion of the food to be examined
(e) A person during pregnancy (including the case that there is a possibility of it) or a person of lactation period
(f) A person who made a blood component donation or a whole blood donation of 200 ml between the month before the start of this examination and the start of the examination.
(g) A person who made a whole blood donation of 400 ml between the month which was 4 months before the start of the examination and the start of the examination.
(h) A person, of which total amount of the collected blood between the month which was 12 months before the start of the examination and the start of the examination, and blood amount planned in this examination exceeds 800 ml.
(i) A person participating in any other examination or a person who is within 4 weeks after the termination of any examination which is the same design as that of this examination.
(j) Others, e.g., a person who was judged as to be inadequate as the subject of this examination by a responsible official doctor or a doctor in charge of this examination.

### 2. Food to be Examined

### 2-1. Contents of Food to be Examined

The food to be examined is in a form of capsule. It contains the following components at the following weights per 1 capsule.

| | | |
|---|---|---|
| Extract powder of cultured | 300 | mg |
| mycelia of Cordyceps militalis | | |
| (same as CM-A) | | |
| Silicon dioxide | 6.8 | mg |
| Calcium Stearate | 17 | mg |
| Potato starch | 16.2 | mg |

### 2-2. Amount to be Ingested

In light of the examination results which were obtained by using mice, the amount of the extract powder from the cultured mycelia of Cordyceps militalis per 1 day, which would be administered to human, was decided to be 25 mg/kg-body weight. In the case where a human having a body weight of 60 kg, the amount is 1,500 mg/day. Therefore, the inventors decided that they ensured 3 capsules of the above capsule (containing 300 mg of the extract powder of cultured mycelia of Cordyceps militalis) ingest in the morning and in the evening (the amount of ingestion of the extract powder from cultured mycelia of Cordyceps militalis per 1 day: 1,800 mg).

It has been confirmed that by the analysis in General Incorporated Foundation Japan Food Research Laboratories, this food to be examined does not contain components harmful to the human body.

### 2-3. Method for Ingestion and Ingestion Period

(Method for ingestion) Within 30 minutes after breakfast and within 30 minutes after dinner, the inventors ensured 3 capsules per/once ingest.
(Ingestion period) Preliminary examination was 1 week, and actual examination was 4 months (16 weeks).

### 3. Examination Design

It was open examination (unblinded experiment).

### (Preliminary Examination)

The inventors ensured the food to be examined ingest for 1 week to 10 subjects (4 women, 6 men; average age: 45.1±30.6).

### (Actual Examination)

The inventors ensured the food to be examined ingest for 4 months (16 weeks) to 13 subjects (9 women, 4 men; average age: 38.4±16.2).

### 4. Collection of Blood and Examination

### 4-1. Collection of Blood

### (Preliminary Examination)

At immediately before the start of the examination and at the end of the examination, blood was collected from the vein

### (Actual Examination)

At immediately before the start of the examination, at 1 month (4 weeks), 2 months (8 weeks) and 4 months (16 weeks) after the start of the examination, blood was collected from the vein.

### 4-2. Examination

The inventors asked the blood examinations to LSI Mediense Corporation. The examination items were blood count (number of erythrocyte, hemoglobin concentration, hematocrit value), concentration of erythropoietin, total cholesterol, TG (neutral fat), HDL-cholesterol, urea nitrogen, uric acid, sodium, potassium, chlorine, calcium, magnesium, serum iron, unsaturated iron binding capacity, CRP (C-reactive protein), AST (GOT), γ-GT (γ-GTP), whole protein and CK (creatine kinase).

Body height, body weight and BMI were also determined.

### 5. Results

### 5-1. Statistical Analyses of Results

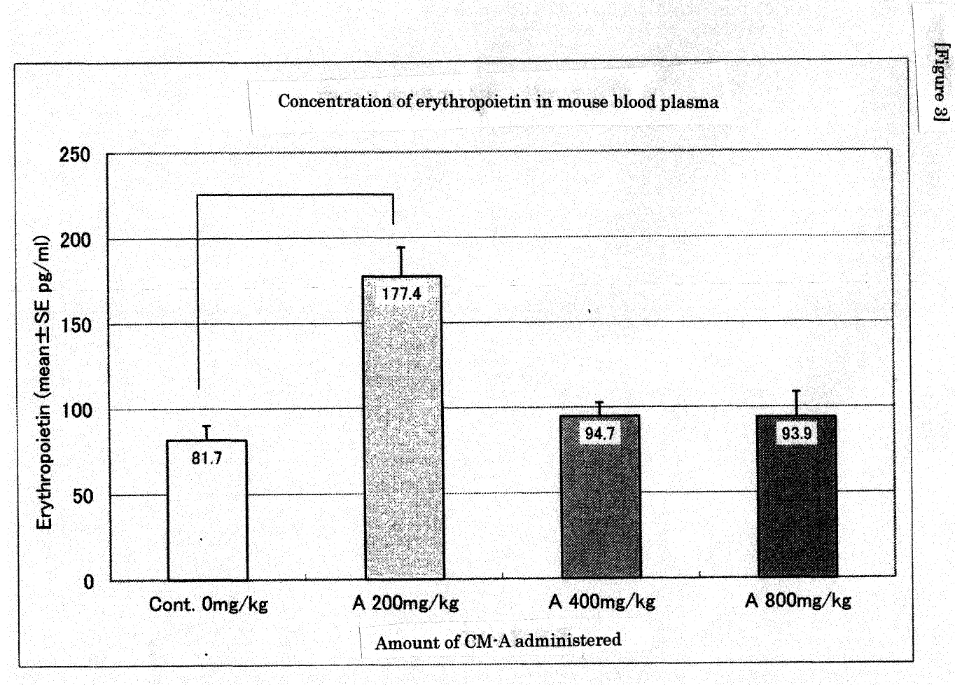

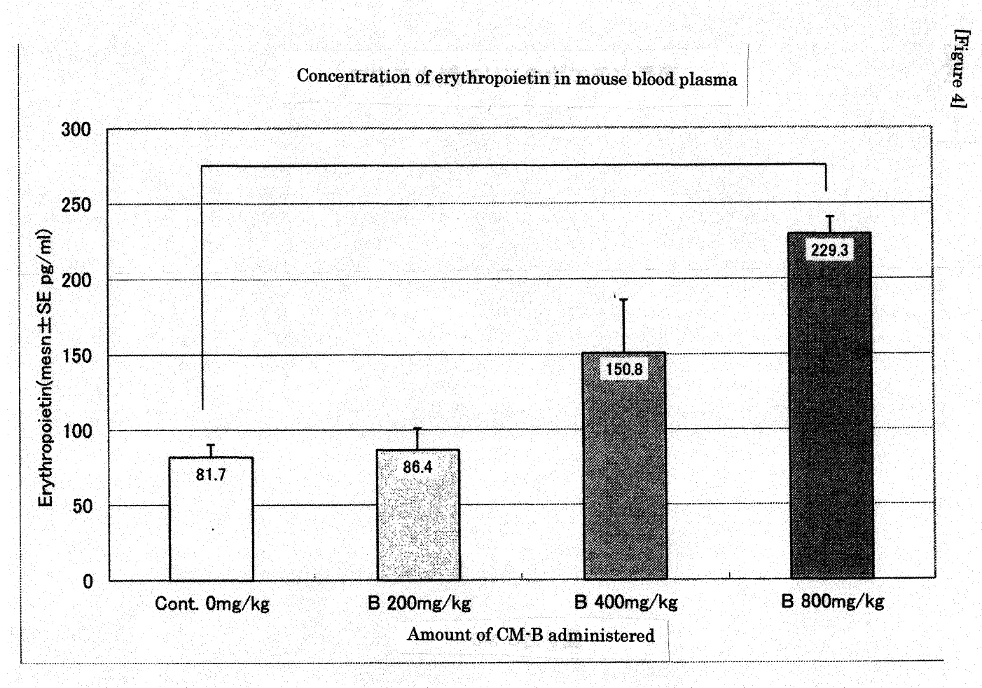

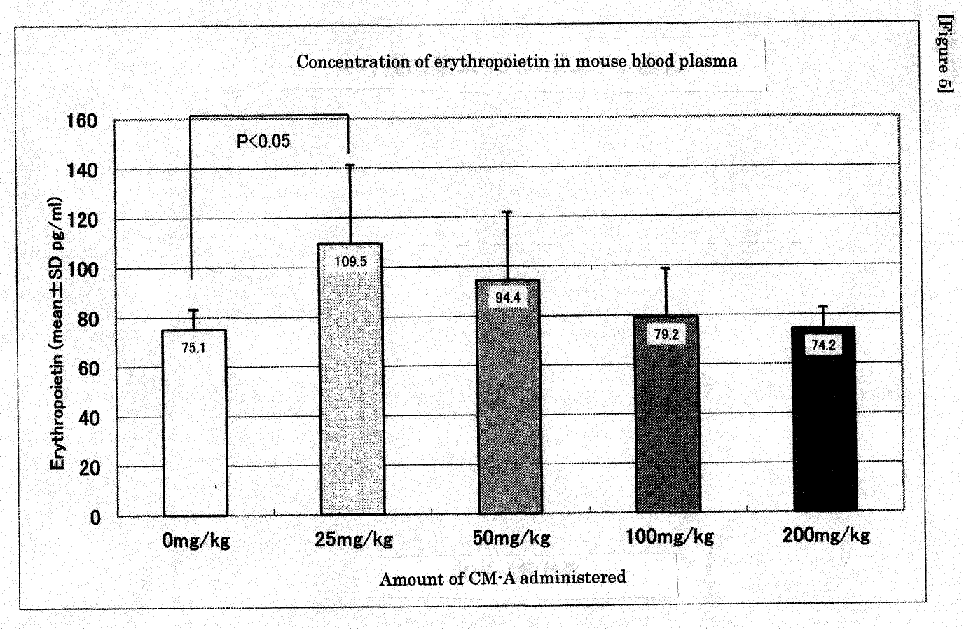

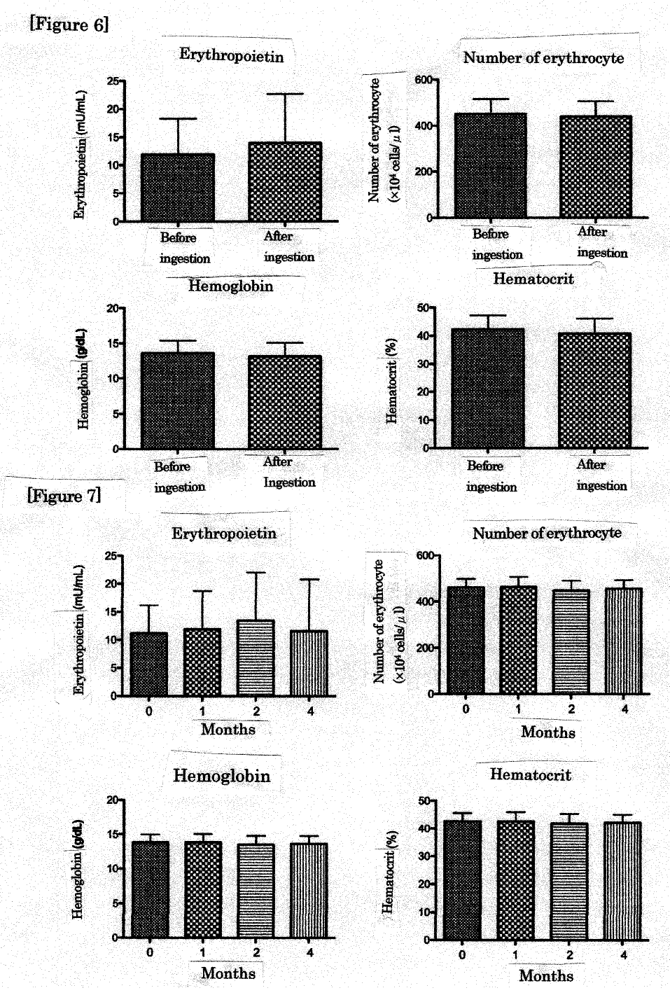

FIG. 6 (preliminary examination) and FIG. 7 (actual examination) show the results of examinations of erythrocyte (concentration of erythropoietin, number of erythrocyte, hemoglobin concentration and hematocrit value). The results were shown by “average±standard deviation.” Comparison between 2 groups was tested by t-test and comparison of 3 groups or more was tested by analysis of variance (ANOVA). When p is less than 0.05 (p<0.05), it was evaluated as to be significantly different.

### 5-2. Results

### (Preliminary Examination)

According to the result of examination of erythrocyte, there was a tendency that erythropoietin concentration increased by ingestion of the food to be examined, although the variation was within a criterial value and there was no statistically significant difference. On the other hand, number of erythrocyte, hemoglobin concentration and hematocrit value decreased with significant difference (p<0.05), although the variations were poor within their criterial values.

### (Actual Examination)

There was a tendency that erythropoietin concentration increased little by little from 1 month after the start of the examination to 2 month after the start of the examination by ingestion of the food to be examined, although the variation was poor within a criterial value and there was no significant difference. On the other hand, number of erythrocyte, hemoglobin concentration and hematocrit value were within criterial values and significant differences did not occur,

### 5-3. Adverse Event

“Adverse event” means every unfavorable or unintentional indication, symptomatic state or disorder (including abnormal variations of laboratory data), which occurred in subjects who ingested the food to be examined. In this examination, by ingestion of the food to be examined, there were no abnormal variations for aspartate aminotransferase (AST) and γ-glutamyltranspeptidase (γ-GT), which were indexes of hepatic functions, urea nitrogen, which was an index of kidney's functions, creatine kinase (CK) of muscle, metabolic indexes such as total protein, total cholesterol, neutral fat, HDL-cholesterol, uric acid and the like, ions such as sodium, potassium and the like, an inflammatory marker such as CRP, and iron-related indexes such as serum iron, unsaturated iron binding capacity and the like. Also, ingestion of the food to be examined did not affect body height, body weight or BMI.

1. A drug composition for inducing erythropoietin or for prevention or treatment of renal anemia activity comprising culture of Cordyceps militalis or an extract from the culture.

2. The drug composition according to claim 1, wherein the culture of Cordyceps militalis is culture of mycelia.

3. The drug composition according to claim 1, wherein the culture of Cordyceps militalis comprises culture that has been cultured by using solid medium.

4.-13. (canceled)

14. A food or beverage composition for inducing erythropoietin or for prevention of renal anemia comprising culture of Cordyceps militalis or an extract from the culture.

15. The food or beverage composition according to claim 14, wherein the culture of Cordyceps militalis is culture of mycelia.

16. The food or beverage composition according to claim 14, wherein the culture of Cordyceps militalis comprises culture that has been cultured by using solid medium.

17. A method for producing the drug composition of claim 1, or the food or beverage composition of claim 14, comprising step (I) and step (II), wherein the step (I) comprises inoculating spawn of Cordyceps militalis to solid medium comprising a protein and a grain and culturing mycelia, and the step (II) comprises obtaining an extract comprising a component that has an erythropoietin-inducing activity from solid culture of Cordyceps militalis that has been obtained through the step (I).

18. The method according to claim 17, wherein the step (II) comprises a step comprising carrying out warming extraction and heating extraction by using ethanol having a concentration of 20 to 50% by weight.

19. The method according to claim 17, wherein the protein comprises a protein derived from a mite's body.

20. A method for producing the drug composition of claim 1, or the food or beverage composition of claim 14, comprising step (1) comprising inoculating spawn of Cordyceps militalis to liquid medium comprising glucose, yeast extract, rice bran (KOMENUKA), sake lees, soybean powder, sodium aspartate, an inorganic substance and water and culturing mycelia.

21. The method according to claim 20, which further comprises step (2) comprising obtaining an extract comprising a component that has an erythropoietin-inducing activity from liquid culture of Cordyceps militalis that has been obtained through the step (1).

22. The method according to claim 21, wherein the step (2) comprises a step comprising adding ethanol to liquid culture of Cordyceps militalis that has been obtained through the step (1) and carrying out warming extraction and heating extraction, wherein the ethanol concentration of liquid comprising the liquid culture and the ethanol is 20 to 50% by weight.
